(19) United States
(12) Patent Application Publication
(10) Pub. No.: US 2013/0317350 A1
(43) Pub. Date: Nov. 28, 2013

(54) USE OF MAGNETIC VORTEX CORES IN MAGNETIC RESONANCE IMAGING AND TUMOR TREATMENT

(71) Applicants: Haiming Fan, Singapore (SG); Jun Ding, Singapore (SG)

(72) Inventors: Haiming Fan, Singapore (SG); Jun Ding, Singapore (SG)

(21) Appl. No.: 14/000,075

(22) PCT Filed: Feb. 15, 2012
(86) PCT No.: PCT/SG2012/000046

§ 371 Date: Aug. 16, 2013

Related U.S. Application Data
(60) Provisional application No. 61/443,887, filed on Feb. 17, 2011.

(51) Int. Cl. A61K 49/18; A61N 2/00

(52) U.S. Cl. CPC A61K 49/18; A61N 2/002
USPC 600/420; 600/12; 600/1

(57) ABSTRACT

Disclosed are a method for enhancing contrast of magnetic resonance imaging and a method for treating tumor.

### RELATED APPLICATION

This application claims priority of U.S. Provisional Application No. 61/443,887, filed on. Feb. 17, 2011. The prior application is incorporated herein by reference in its entirety.

### BACKGROUND OF THE INVENTION

Superparamagnetic nanoparticles dispersions have been widely used in bio-medical applications, e.g., magnetic resonance imaging (MRI), magnetic hyperthermia, and drug delivery. Nanoparticles in such suspensions usually have diameters below 10 nm so as to achieve superparamagnetism and overcome sedimentation against Brownian motion and inter-particulate magnetic interactions. Yet, it is difficult to retain the stoichiometry, size uniformity, and magnetism of nanoparticles of these dimensions. There is a need to improve stability.

### SUMMARY OF THE INVENTION

This invention is based, at least in part, on unexpected findings that magnetic vortex core (MVC) in a nanoring shape each with a size over 20 nm form a stable magnetic fluid and that such a fluid has a high saturation magnetization and a high magnetic susceptibility.

Accordingly, one aspect of this invention features a method of enhancing MRI contrast. This method includes administering to a subject in need thereof an effective amount of an MVC nanoring.

Examples of the MVC nanoring include, but are not limited to, Fe₃O₄, γ-Fe₂O₃, and MFe₂O₄ nanorings (M=cobalt ion, nickel ion, copper ion, or manganese ion), all of which can be further modified to form quantum dot-capped MVC nanorings. Their sizes are 20-300 nm in height, 35-1000 nm in outer diameter, and 12-750 nm in inner diameter. More preferably, the sizes are 20-150 nm in height, 35-250 nm in outer diameter, and 12-150 nm in inner diameter.

Another aspect of this invention features a method of treating tumor. This method includes delivering to a tumor site in a subject an effective amount of an MVC nanoring, placing the tumor site in an alternating magnetic field (AMF), and maintaining the tumor site in the AMF for a pre-determined duration of time so as to kill tumor cells.

The above-described treatment method can be combined with radiotherapy or chemotherapy.

The details of one or more examples of the invention are set forth in the description below. Other features, objects, and advantages of the present invention will be apparent from the following the detailed description and drawings, and also from the appended claims.

### DESCRIPTION OF DRAWINGS

FIG. 1 is a schematic illustration for a typical synthesis of free standing MVC nanorings (FVIOs).

FIG. 2 is a schematic illustration for the synthesis of water-dispersible quantum dot-capped MVC nanorings (QD-FVIOs). The branched PEI is drawn as line for simplification.

FIGS. 3A and 3B are TEM images of QD-FVIO1 and QD-FVIO2, respectively, FIG. 3C is a HRTEM image of QD-FVIOs, and FIG. 3D is an EDS spectrum of QD-FVIOs. The insets in FIGS. 3A and 3B are the high magnification TEM images of single QD-FVIOs (scale bar, 50 nm).

FIG. 4A includes photo images of QD-FVIOs in aqueous solution taken (I) in ambient condition, (II) under UV radiation, and (III) under both UV radiation and external magnetic field; FIG. 4B is a diagram showing hydrodynamic diameters of QD-FVIOs; FIG. 4C is a diagram showing the effect of salt concentration on fluorescence signal variations of QD-FVIOs; and FIG. 4D is a diagram showing the effect of pH in water solution on fluorescence signal variations of a QD-FVIO.

FIG. 5A is a diagram showing hysteresis loops of QD-FVIOs at 300 K, FIG. 5B is an off-axis electron hologram of a single QD-FVIO, FIG. 5C is a diagram showing direction of the magnetic induction under field-free conditions following magnetization indicated by color as shown in the color wheel (red=right, yellow=down, green=left, blue=up), and FIGS. 5D and 5E are schematic illustrations of vortex/onion state in QD-FVIOs dispersion in the absence/presence of external magnetic field. Artows indicate the spin direction.

FIG. 6A is a typical absorption spectrum of QD-FVIOs at room temperature; FIG. 6B shows typical time-resolved fluorescence spectra for PEI-QDs and QD-FVIOs; FIG. 6C shows two-photon emission spectra of multicolor QD-FVIOs excited by 800 nm, 100 fs laser pulses; and FIG. 6D is a diagram showing typical power dependent photoluminescence intensity of QD-FVIOs.

FIG. 7 shows images of in vitro T2* weighted MRI of QD-FVIOs in 2% agarose and commercial ferucarbotran in water.

FIGS. 8A and 8B are histograms showing cell viabilities in the presence of QD-FVIO1 and QD-FVIO2, respectively, at various Fe₃O₄ concentrations.

FIG. 9 is a diagram showing hyperthermia effects of 70 nm MVC nanorings at a field of 27 kA/m, 400 kHz.

### DETAILED DESCRIPTION OF THE INVENTION

The present invention features use of a ferrofluid, containing MVC nanorings, to (1) enhance MRI contrast for detecting pathological lesions and (2) enhance hyperthermic effect for treating cancer.

MRI is a noninvasive clinical imaging technique used in radiology to visualize detailed internal structure. It provides much greater contrast between different soft tissues of the body than computed tomography. Thus, MRI is especially useful in neurological (brain), musculoskeletal, cardiovascular, and oncological (cancer) imaging.

MRI constructs images using the inherent natural abundance and the nature of the proton spins of water molecules in human bodies. In addition to the proton density, two major mechanisms contribute to the degree of contrast enhancement, i.e., the longitudinal (T₁) and transverse (T₂ and T₂*) relaxation times.

MRI contrast agents have been used to improve the visibility of internal body structures by altering the relaxation times of tissues and body cavities where they are present. They can be divided into two major types, i.e., positive and negative contrast agents. Positive contrast agents act to shorten mainly T₁ and at the same time provide moderate impact on T₂, thus generating a bright image. Negative contrast agents, on the other hand, mainly shorten T₂ and and T₂*and lead to signal reduction, i.e., a dark image.

Improved differentiation between fat (brighter) and water (darker) is achieved by reduction of T₁ relaxation time, which increases T₁ signals. Brain scan contrast between white matter and grey matter can be improved by reduced T₂ relaxation time, which reduces T₂ and T₂* signals.

Relaxivity (r₁, r₂, or r₂*) is a measure of the efficacy of the MRI contrast agents in shortening the time for the relaxation processes at 1 mM concentration, and a large value of relaxivity usually reflects a better in vivo performance. The alternation of the relaxation rate (r₁, r₂, or r₂*) is governed by magnetic properties of these contrast agents. Efforts have constantly been made to improve the image resolution of MRI scans by identifying positive contrast agents that are highly paramagnetic.

The most commonly used compounds for positive contrast enhancement are gadolinium-based. Chelated gadolinium contrast agents enhance imaging vessels in MR angiography or brain tumors associated with the degradation of the blood-brain barrier by enhancing the T₁ signals. However, they cause nephrogenic systemic fibrosis in patients with severe kidney failure.

Alternatively, manganese chelates, such as Mn-DPDP, enhance the T₁ signals and have been used for the detection of liver lesions.

Two types of nano-sized iron oxide contrast agents exist: superparamagnetic iron oxide (SPIO) and ultrasmall superparamagnetic iron oxide (USPIO). They are frequently used for negative contrast agents due to their superparamagentism and high biocompatibilities. Both contrast agents, when injected during imaging, reduce the T₂ signals of absorbing tissues. However, the nanoparticle size in suspension has to be small enough, i.e., below 10 nm in diameter, to overcome sedimentation against Brownian motion and inter-particulate magnetic interactions. The SPIO and USPIO nanoparticles themselves are not stable and have difficulties to retain their stoichiometry, size uniformity and magnetism, which, in turn, results in rather poor enhancement of MR signals.

Described herein is a method of using a MVC nanoring as a high-performance MRI contrast agent. MVC nanorings each have a closed domain structure without magnetic poles or stray fields. Their sizes are 20-300 nm in height, 35-1000 nm in outer diameter, and 12-750 nm in inner diameter. The outer diameter is always larger than the other two dimensions, i.e., height and inner diameter.

The advantages of a MVC nanoring contrast agent are: (1) weak inter-particular magnetic interactions as compared to nonhollow-shaped magnetic materials; (2) formation of a well-dispersed ferrofluid as long as sizes of nanorings or nanotubes do not exceed 300 nm in height and width; (3) a wide size range of nanorings or nanotubes, i.e., varying from 20 nm to 300 nm in height and width (much larger as compared to that of spherical SPIO nanoparticles, i.e., under 10 nm in diameter); (4) a high saturation magnetization and enhanced remanent magnetization;(5) it can alter the MRI signal of a dimension larger than its size (6) a high magnetic susceptibility so that magnetic saturation, leading to a fast and strong response, can be easily reached in a relatively low external magnetic field; and (7) high stability with a large surface area for convenient surface modification and functionalization, which can be used for drug delivery and other applications.

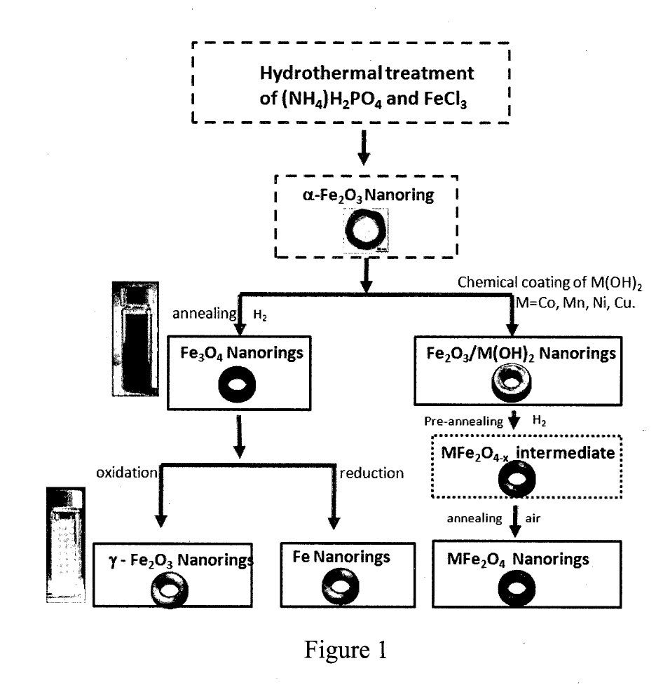

As shown in FIG. 1, α-Fe₂O₃ nanorings or nanotubes with desired sizes (including height and diameters) can be synthesized through hydrothermal treatment of NH₄H₂PO₄ and FeCl₃ with optimized experimental parameters, such as temperature, concentrations of reactants, etc. Fe₃O₄ nanorings or nanotubes is then synthesized through hydrogen reduction. If a higher stability is required, Fe₃O₄ nanorings or nanotubes can be oxidized into γ-Fe₂O₃. With a higher reduction temperature, Fe₃O₄ nanorings or nanotubes can be reduced into Fe. However, pure Fe can be easily oxidized into iron oxide.

Other metal elements, e.g., M=Ni, Mn or Co, may be used to fine-tune magnetic properties to stabilize the above-described vortex structure. For example, Ni in the form of Ni₁₋ₓFe₂₊ₓO₄ can reduce magnetocrystalline anisotropy and saturation magnetization; and Co can, on the other hand, enhance anisotropy and increase saturation magnetization and corrosion resistance.

Nanorings can be further, modified to produce quantum dot capped magnetite nanorings (QD-FVIOs). Through electrostatic interaction, cationic polyethylenimine (PEI) capped QD have been firmly graft into negatively charged MVC nanorings modified with citric acid.

Unexpectedly, experimental results demonstrated that the above-described QD-FVIOs enable significant reductions of inter-particular interactions in magnetic fluid and offer great enhancement effects on shortening T₂*, resulting in a large r₂* relaxation rate and r₂*/r₁ ratio that are 4 times and 110 times higher than that of SPIO nanoparticles, e.g., Ferucarbotran (Resovist; Schering, Berlin, Germany). The r₂* relaxivity of FVIO is the largest one so far reported in the field. These unexpected results provide a new approach (rather than increasing the particle number or the particle size) to achieve significant enhancement of MRI signals in T₂* weighed sequences.

In addition to be MRI contrast agents, MVC nanorings or nanotubes can also be used as hyperthermic agents for cancer therapy. Magnetic fluid or ferrofluid has been utilized for the magnetic induction hyperthermia (MIH) to treat cancers. The principle is that, when magnetic fluids are delivered to tumor sites and subjected to an alternating magnetic field (AMF), the magnetic particles in the fluid produce damaging heat from an alternating magnetization loss and kill the cancer cells at a controlled temperature of 43-56° C. Magnetic susceptibility plays an important role in performance of magnetic hyperthermic agents, containing nanoparticles. MVC nanorings possess high saturation magnetization, which can be easily reached in a relatively small field due to their high magnetic susceptibility. This high and easily-reached saturation magnetization results in a high magnetization loss when an alternating magnetic field is applied to these MVC nanorings. Indeed, high damaging heat is produced by a fluid containing MVC nanorings, when the fluid is subjected to an alternating magnetic field. Therefore, the high magnetic susceptibility of MVC nanorings, together with their high saturation magnetization, enhances hyperthermia effect on tumor.

The present invention thus also provides a method of using a MVC nanoring to treat tumor in a subject. This method includes delivering an effective amount of a MVC nanoring to a tumor site in a subject, placing the tumor site in an AMF, and maintaining the tumor site in the AMF for a pre-determined duration of time so as to kill tumor cells. The AMF has a frequency of 100-500 kHz.

As used herein, the term “tumor” refers to proliferating malignant or nonmalignant disease. Proliferating malignant disease refers to melanoma, Kaposi's sarcoma, osteosarcoma, neuroblastoma, rhabdomyosarcoma, Ewing's sarcoma, Soft tissue sarcoma, skin cancer, lymphoma, breast cancer, germ cell tumor, primitive neuroectodermal tumor, brain glioma, brain meningioma, head and neck cancer, thyroid cancer, thymic cancer, cervical cancer, anus cancer, colorectal cancer, prostate cancer, kidney cancer, lung cancer, hepatocellular carcinoma, cholangiocarcinoma, stomach cancer, pancreatic cancer, esophageal cancer, or a virus-associated tumor.

The proliferating nonmalignant disease refers to orbital pseudotumor, keloid, wart, keratoacanthoma, hemangioma, arteriovenous malformation, bursitis, desmoid tumor, ameloblastoma, heterotopic bone formation, or adenoma.

The term “an effective amount” refers to the amount of a MVC nanoring or nanotube that is required to confer the intended effect in the subject. Effective amounts may vary, as recognized by those skilled in the art, depending on route of administration, excipient usage, and the possibility of co-usage with other agents.

To practice the method of this invention, the above-described contrast agents can be administered orally or parenterally, depending on the subject of interest. The term “parenteral” as used herein includes subcutaneous, intravenous, intramuscular, or intrasternal injection.

Oral administration is well suited to enhance the gastrointestinal tract scans, while intravascular administration proves more useful for enhancing the appearance of blood vessels, tumors, or inflammation. Contrast agents may also be directly injected into a joint in the case of artgrigrans, MR images of joints.

A sterile injectable composition, e.g., a sterile injectable aqueous and oleaginous suspension, can be formulated according to techniques known in the art using suitable dispersing or wetting agents (such as Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. Other commonly used surfactants such as PEG derivatives, PLGA derivatives, Tweens or Spans, other similar emulsifying agents, or bioavailability enhancers which are commonly used in the manufacture of pharmaceutically acceptable liquid can also be used for the purposes of formulation.

A composition for oral administration can be any orally acceptable dosage form including, but not limited to, aqueous suspensions, dispersions, and solutions. If desired, certain sweetening, flavoring, or coloring agents can be added.

A “subject” refers to a human and a non-human animal. Examples of a non-human animal include all vertebrates, e.g., mammals, such as non-human primates (particularly higher primates), dog, rodent (e.g., mouse or rat), guinea pig, cat, and non-mammals, such as birds. In a preferred embodiment, the subject is a human. In another embodiment, the subject is an experimental animal or animal suitable as a disease model. A subject to be treated for the above-described disorder can be identified by standard diagnosing techniques for the disorder.

“Treating” refers to administration of a MVC ring to a subject, which has one of the above-mentioned disorders, with the purpose to cure, alleviate, relieve, remedy, or ameliorate the disorder, the symptom of the disorder, the disease state secondary to the disorder, or the predisposition toward the disorder. The treatment method can be performed alone or in conjunction with other drugs or therapy.

Also described herein is a method of cellular imaging. This method includes contacting the thus prepared QD-FVIO with a cell and imaging with two-photon fluorescence image.

To facilitate understanding of this invention, we have attached the following three articles entitled “Quantum Dot Capped Magnetite Nanoring as High Performance Nanoprobe for Multiphoton Fluorescence and Magnetic Resonance Imaging;” “Shape-Controlled Synthesis of Single-Crystalline Fe₂O₃ Hollow Nanocrystals and Their Tunable Optical Properties;” and “Single-Crystalline MFe₂O₄ Nanotubes/Nanorings Synthesized by Thermal Transformation Process for Biological Applications.”

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever.

All publications/documents cited herein or attached hereto are incorporated by reference in their entirety. Furthers any mechanism proposed below does not in any way restrict the scope of the claimed invention, nanorings or nanotubes

### Materials and Methods

### Preparation of α-Fe₂O₃ Nanorings or Nanotubes

The α-Fe₂O₃ nanorings or nanotubes were synthesized by a hydrothermal treatment of FeCl₃ solution in the presence of NH₄H₂PO₄. In a typical experimental procedure for nanotubes (having an average size of 260 nm in height, 98 nm in outer diameter, and 82 nm in inner diameter), 1.60 ml of aqueous FeCl₃ solution (0.5 M) and 1.44 ml of aqueous NH₄H₂PO₄ solution (0.02 M) were mixed with vigorous stirring. Distilled water was then added to a final volume of 40 ml. After stirring for 10 minutes, the mixture was transferred into a Teflon-lined stainless-steel autoclave with a capacity of 50 ml for hydrothermal treatment at 220° C. for 60 h. The autoclave then cooled down to room temperature naturally, a red precipitate was separated by centrifugation, washed with distilled water and absolute ethanol, and dried under vacuum at 80° C. The obtained sample was labeled S1. By simple adjustment of the reactant concentration, different-sized α-Fe₂O₃ hollow nanocrystals were prepared. For sample S2 (having an average size of 50 nm in height, 74 nm in outer diameter, and 60 nm in inner diameter), 0.8 ml of aqueous FeCl₃ solution (0.5 M) and 0.72 ml of aqueous NH₄H₂PO₄ solution (0.02 M) were used. Also, 0.4 ml of aqueous FeCl₃ solution (0.5 M) and 1.42 ml of aqueous NH₄H₂PO₄ solution (0.02 M) were used for the preparation of sample S3 (having an average size of 10 nm in height, 150 nm in outer diameter, and 117 nm in inner diameter). All of the reagents were of analytical purity and purchased from Sigma-Aldrich Chemical, Co.

### Preparation of Fe₃O₄ and γ-Fe₂O₃ Nanorings or Nanotubes

Fe₃O₄ and γ-Fe₂O₃ nanorings and nanotubes were prepared by direct solid-gas reaction. In brief, 0.5 g of α-Fe₂O₃ nanorings or nanotubes was heated in a horizontal quartz tube furnace at 420° C. for 2 h under a constant flow of 5% H₂/95% Ar at 800 sccm (standard cubic centimeter per minute). Then the furnace was cooled down to room temperature without any changes in atmosphere. The black Fe₃O₄ product was collected from the small quartz. Annealing of the thus prepared Fe₃O₄ nanorings or nanotubes in an open tube furnace at 280° C. for 2 h yields γ-Fe2O₃ nanorings or nanotubes.

Preparation of M₁₋ₓFe₂₊ₓO₄ Nanorings or Nanotubes (M═Ni, Mn, or Co Ion)

M₁₋ₓFe₂₊ₓO₄ nanorings or nanotubes were synthesized through introducing a metal ion into single-crystalline α-Fe₂O₃ nanoring or nanotube templates. Metal hydroxides deposited on the surface of α-Fe₂O₃ nanorings or nanotubes by wet chemical precipitation method are used as a source for metal ions, and they are attached to α-Fe₂O₃ through the olation and oxolation bridges in alkaline condition. To prepare MFe₂O₄ nanorings (i.e., x=0), the initial molar ratio of a metal ion (M) and Fe³⁺ in the reaction solution is about 0.63.

In a typical procedure of preparing CoFe₂O₄ nanorings or nanotubes from α-Fe₂O₃, 2.5 mL of 0.1 M CoSO₄ solution was added into 20 mL of 0.01 M α-Fe₂O₃ aqueous suspension. The mixture was heated to 60° C. with magnetic stirring, and then 40 mL of 0.01 M NaOH was added into the solution under vigorous stirring. The stirring was kept for 30 min at 60° C. After the reaction was completed, the α-Fe₂O₃/Co(OH)₂ core/shell precipitation was separated by centrifugation and dried at 60° C. The resultant Fe₂O₃/Co(OH)₂ core/shell sample was first annealed for 30 min under a constant flow of 5% H₂/95% Ar at 800 sccm at 300° C., and then annealed in air at 720° C. for 3 h to activate the interfacial solid-solid reaction. MnFe₂O₄ and NiFe₂O₄ were prepared similarly, using manganese acetate and nickel chloride, respectively, as reactants. All the reagents were of analytical purity purchased from Sigma-Aldrich Co. The resultant molar ratio of M and Fe in the collective nanotubes is 0.56±0.04.

### Preparation of Citric Acid-Capped Fe₃O₄ Nanoring Aqueous Solution

In order to examine the size effect on the MRI signal, Fe₃O₄ nanorings with average outer diameters of 162 nm (labeled FVIO1) and 72 nm (labeled FVIO2) respectively were prepared. The obtained single-crystal Fe₃O₄ nanorings have a narrow size distribution (<10%). The ring axis is in the [111] or [112] direction of Fe₃O₄. The water-soluble Fe₃O₄ nanorings were obtained by further modifying the magnetite surface with citric acid. In a typical procedure, Fe₃O₄ nanorings were dispersed into water by an ultrasonication with a concentration of 2 mg/ml. The pH of the aqueous solution was adjusted to 3.0 by HCl (0.1 M). Then citric acid (5% molar ratio of Fe) was added to the suspension under magnetic stirring. After 4 h of stirring, the suspension was washed with water by magnetic decantation three times and Fe₃O₄ nanorings were redispersed into water with a concentration of 1 mg/ml.

### Preparation of PEI Capped CdSe/ZnS Core/Shell Nanocrystals

The multicolor trioctylphosphine oxide (TOPO) capped CdSe@ZnS QDs were prepared by a well-established organometallic synthetic approach method as described in Hashizume, et al., J. Lumin. 2002, 98, 49-56; and Fan, et al., Appl. Phys. Lett. 2007, 90 021921. These QDs exhibit a high quantum efficiency (above 50%) at room temperature as well as a narrow size distribution (<5%). In order to prepare the polyethyleneimine (PEI) coated QDs, 100 mg of PEI (branched, Mw 150 000, 50% w/v) was first dissolved in 50 ml of absolute ethanol. Then 2 ml QDs chloroform solution (50 mM) was mixed with 18 ml of PEI (20 mg/ml) ethanol solution, followed by an ultrasonic treatment for 20 min. After that, the mixture was kept under magnetic stirring for 2 h at room temperature until the ligand exchange reaction was completed. The obtained PEI capped QDs (PEI-QDs) were stored at 4° C.

### Preparation of PEI Quantum Dot Capped Fe₃O₄ Nanorings

An equal volume of PEI-QDs solution (0.5 mM) was added as drops into 10 ml citric acid-capped Fe₃O₄ nanoring aqueous solution (0.1 mg/ml Fe₃O₄, pH=4.0) under ultrasonication. After ultrasonic treatment for 30 min, the mixture was kept under magnetic stirring for 4 h at room temperature. The final product of QD capped magnetite nanorings (QD-FVIOs) was separated and washed with water three times by magnetic decantation. About 45-65% of the PEI-QDs were effectively absorbed on Fe₃O₄ nanorings, determined by the absorption spectra of PEI QDs before and after the conjugation process. The estimated ratios of QDs and magnetite nanorings are about 1.8×10³ QD particles per nanoring (QD-FVIO1) and 3.6×10² QD particles per nanoring (QD-FVIO2), respectively.

Preparation of a Phosphorylated mPEG (P-MPEG)-Capped Fe₃O₄ Nanoring Aqueous Solution

P-MPEG was synthesized by reacting POCl₃ with mPEG by a well-established synthetic approach described in Tromsdorf et al, Nano Lett. 2009, 9 (12), 4434-4440. The Fe₃O₄ nanoring were first dispersed into chloroform. 50 msg phophorylated mPEG was added into a 0.5 nM nanoring chloroform solution. The mixture was under shaking for 2 h under ambient condition. After the complete of ligand exchange reaction, the solvent was evaporated under a flow of argon. The deposition was re-dispersed in water.

### Characterization of QD-FVIOs

Transmission electron microscopy (TEM) and high-resolution TEM (HRTEM) analysis, energy-dispersive X-ray spectroscopy (EDS) were performed with a field-emission transmission electron microscope (JEOL, JEM 2010, accelerating voltage 200 kV). Magnetic properties were measured using a MPMSXL-5 Quantum Design superconducting quantum interference device (SQUID) magnetometer with a field up to 5 T. To investigate the multiphoton excited photoluminescence (PL) properties of these QD-FVIOs, a Coherent Legend regenerative amplifier (seeded by a Mira) (200 fs, 1 kHz, 800 nm) was used as the excitation source. The laser pulses were focused by a lens (f=30 cm) on the sample solutions in a 2-mm-thick quartz cell (beam spot ˜1 mm inside the cell). The emission from the QD-FVIOs was collected at a backscattering angle of 150° by a pair of lenses and optical fibers, and directed to a spectrometer (Acton, Spectra Pro 2500i coupled CCD Princeton Instruments, Pixis 400B). A short-pass filter with a cutoff wavelength of 730 nm was placed before the spectrometer to minimize the scattered excitation light. The time-resolved upconversion PL measurements were collected using an Optronis Optoscope™ streak camera system which has an ultimate temporal resolution of 6 ps. The absorption of QD-FVIOs was measured using a Shimadzu UV-1700 spectrophotometer.

### In Vitro Relaxivities of MRI

Particle relaxivities (r₁, r₂, and r₂*) were obtained from MRI of in vitro QD-FVIOs dispersed in 2% agarose using a Siemens Symphony 1.5 T clinical scanner with a head coil as previously described in Lee, et al., Biomaterials 2010, 31, 3296-3306. The control experiment was performed using ferucarbotran in water solution. Fe concentration in agarose was calibrated using inductively coupled plasma spectroscopy (ICP).

### Multiphoton Bioimaging and Cell Uptake

QD-FVIOs for in vitro cell imaging were studied using MGH bladder cancer cells. MGH bladder carcinoma cells were seeded in eight well chamber slides (Labtek II, Nunc, USA) at a seeding density of 5×10⁴ and incubated overnight at 37° C., 5% CO₂. The QD-FVIOs were incubated at a concentration of 0.05 mg/ml in serum-free RPMI medium for 2 h at 37° C. After that, the cells were washed three times with phosphate buffered saline (PBS) and fixed with 2% paraformaldehyde for 15 min. The cells were then rinsed twice with PBS and subsequently mounted with Vectasheild fluorescent mounting medium (Vector laboratories, Burlingame, Calif.). Multiphoton fluorescence images showing the QD-FVIOs labeled MGH cells were captured using two-photon laser scanning confocal microscope with META detector (Carl Zeiss, LSM 510 NLO, Germany). Both an argon ion laser and a Coherent Mira Titanium Sapphire tunable IR laser were used to excite cell autofluorescence (excitation 488 nm) and the QD-FVIOs (excitation 756 nm) respectively. Fluorescence images were taken using a long pass filter of 560 nm. Photomultiplier tube (PMT) gain and offset were adjusted to give subsaturating fluorescence intensity with optimal signal-to-noise ratio. The cells after 24-h incubation with QD-FVIOs through the same procedure were used for the intracellular colocalization studies. The cells were further incubated with early endosome marker at 1:100 (abcam, UK, ab70521) for 1 h at room temperature, followed by incubation with a FITC conjugated secondary antibody 1:1000 (abcam, UK, ab6785) for 1 h. As for TEM investigation, the QD-FVIOs stained cells were fixed in 2.5% glutaraldehyde for 1 h before being washed three times with PBS and osmicated with osmium tetroxide. The samples were then dehydrated with an ascending series of alcohol and embedded in Araldite. Ultrathin sections were cut by a glass knife and doubly stained with uranyl acetate and lead citrate before viewing in a Philips EM280S transmission electron microscope.

### In Vitro Cytotoxicity

In vitro cytotoxicity assay was performed to assess the toxicity of QD-FVIOs on normal and cancer cells. Normal human lung fibroblast cells (NHLF), MGH bladder cancer cells, and SK-BR3 breast cancer cells were used for this experiment. Cell counting kit-8 (CCK-8) (Dojindo Molecular Technologies, Maryland, USA) was used to perform the cytotoxicity assay. Briefly, 100 μl of cell suspension (5000 cells/well) was seeded in a

96-well plate and was preincubated for 24 h in a humidified incubator at 37° C., 5% CO₂. To screen the cytotoxicity of the QD-FVIOs, the cells were treated with different concentrations of QD-FVIOs for 24 h and dimethyl sulfoxide (DMSO) was used as a solvent control. CCK-8 solution was thawed and 10 μl was added to each well of the plates. The plates were then incubated for 4 h. The absorbance was measured at 450 nm using a microplate reader. The number of viable cells for each concentration was compared to a standard curve of known cell density and normalized to the solvent control.

### In Vitro Hyperthermic Effect of Fe₃O₄ Nanorings

Hyperthermic effect of Fe₃O₄ nanorings was measured in the manner as described in Maity et al., 2010, Nanomedicine 5(10): 1571-1584. Briefly, alternating current (AC) magnetic field-induced heating ability of the nanorings was determined from the time-dependent calorimetric measurements using a radiofrequency generator (EASYHEAT-5060, Ameritherm, Scottsville, N.Y., USA) operating at 240 kHz frequency. Next, 1 ml of a phosphorylated mPEG (P-MPEG)-capped Fe₃O₄ nanoring aqueous solution was subjected to 89-kA/m AC field and time-dependent temperature rise was monitored at several time points using an optical fiber-based temperature probe (FLUOTEMP Series, FTP-LN2). The specific absorption rate (SAR) was calculated using the following equation:

$$SAR = C \frac{Δ T}{Δ t} \frac{1}{{m}_{Fe}}$$

where C is the specific heat of solvent (here Cwater=4.18 J/g ° C.), ΔT/Δt is the initial slope of the time-dependent temperature curve and m_{Fe} is weight fraction of magnetic element (i.e., Fe) in the sample.

### Results and Discussion

### Chemical Synthesis and Stability of QD-FVIOs

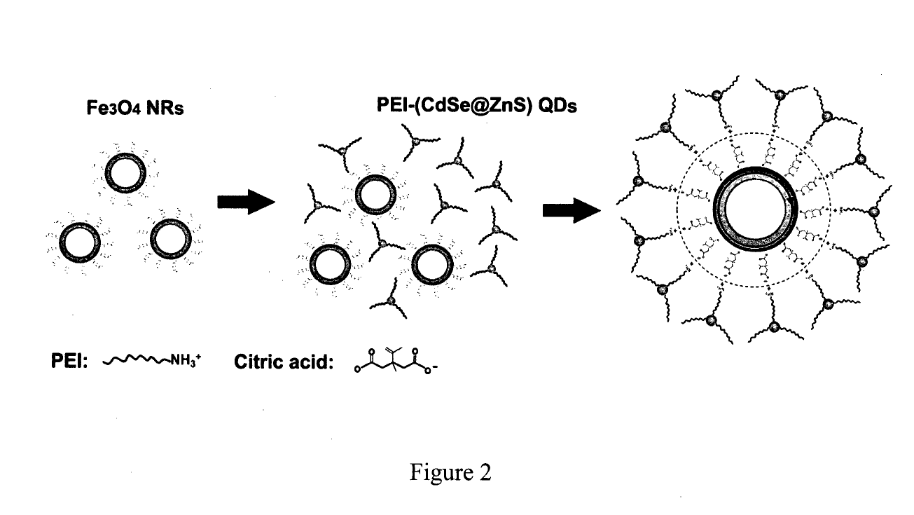

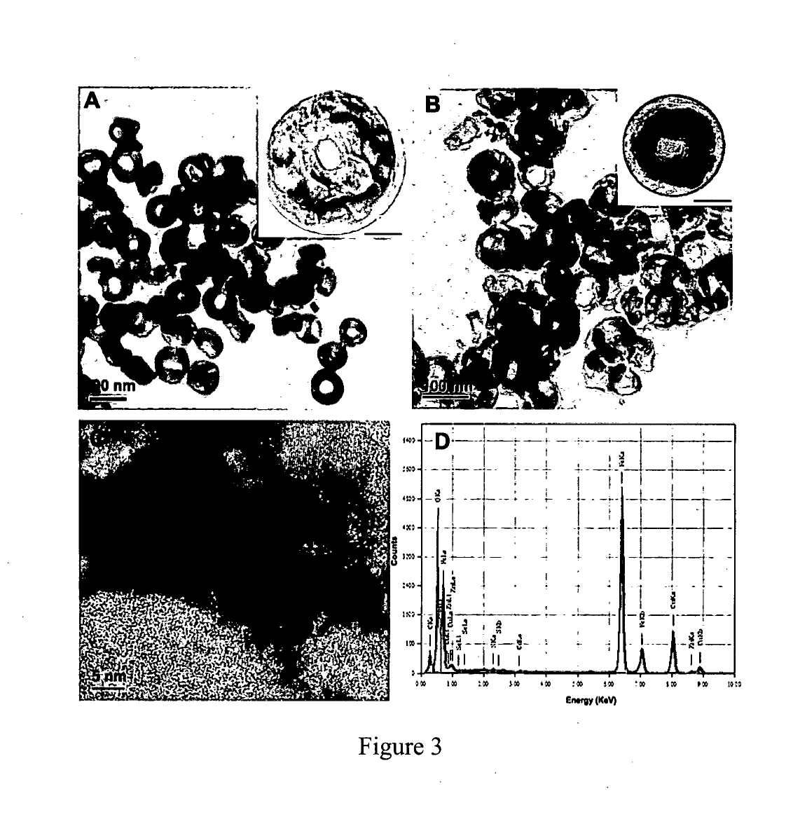

The synthetic strategy of QD-FVIOs is shown in FIG. 2. Electrostatic attraction is the main driving force used for the conjunction of positively charged PEI capped CdSe@ZnS QDs with NH₂ (or NH₃⁺) groups and negatively charged citric acid capped magnetite nanorings with —COOH (or —COO) groups. First, the magnetite nanorings with controlled size were prepared by hydrothermal growth and post-template reduction (Jia, J. Am. Chem. Soc. 2008, 130, 16968-16977; Fan, et al., ACS Nano 2009, 3, 2798-2808; and Fan, et al., J. Phys. Chem. C 2009, 113, 9928-9935) followed by the surface modification of citric acid: Second, highly luminescent polyethyleneimine (PEI) capped CdSe@ZnS QDs were mixed with citric acid stabilized magnetite nanorings under ultrasonication. The QDs were firmly grafted to magnetite narorings through the electrostatic interaction of PEI and citric acid. In comparison with the Fe₃O₄/Au core/shell nanoparticles with single PEI ligand (Goon, et al., Chem. Mater. 2009, 21, 673-681), the presence of citric acid on the surface of magnetite nanorings can enhance the absorption of PEI capped CdSe@ZnS QDs and improve the stability of magnetite nanorings as well (Zhang, et al., Colloids Surf. A 2006, 276, 168-175). The final product of QD-FVIOs was washed with water several times and separated by magnetic decantation. FIGS. 3A and B shows TEM images of as-prepared QD-FVIOs. Two sized QD-FVIOs, QD-FVIO1 and QD-FVIO2 with the sizes of 210±20 nm and 100±10 nm respectively, were synthesized. The particle sizes are slightly larger than bare FVIOs after the conjunction of QDs. The insets in FIGS. 3A and B are the representative single QD-FVIO nanorings. Because of the dense PEI layer, the QDs attached on magnetite nanorings show blurred spots when observed from the high resolution TEM image as shown in FIG. 3C. Energy-dispersive X-ray spectrum (EDS) shown in FIG. 3D confirms the existence of the elements Fe, Cd, and Zn. The Fe/(Cd+Zn) atomic ratio is around 9.6±1, which is roughly consistent with the estimated particles ratio of Fe₃O₄ nanorings and CdSe/ZnS QDs during the synthetic process.

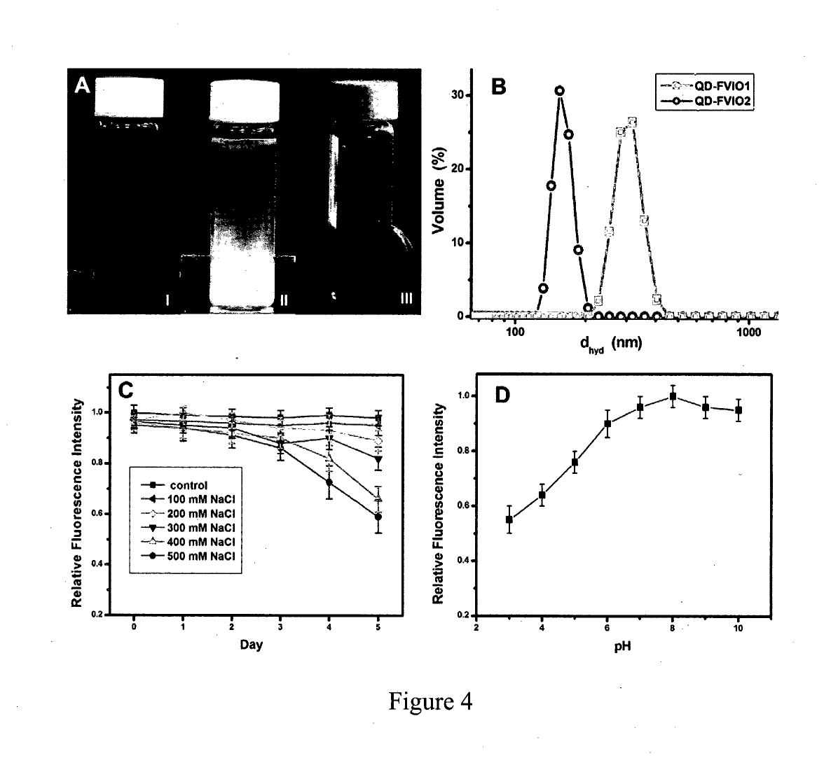

The stability of the QD-FVIOs was investigated using dynamic light scattering and photoluminescence (PL) intensity. The optical images in FIG. 4A show that the obtained QD-FVIOs are water-soluble with minimal or no aggregation, and the strong light emission can be observed under UV radiation. However, the QD-FVIOs will be aggregated under an external magnetic field, which consequently suppresses the emission of QD-FVIOs. As seen in FIG. 4B, the measured hydrodynamic sizes in aqueous solution with a pH of 7 are about 310 nm for QD-FVIO1 and 155 nm for QD-FVIO2, which are slightly larger than that under TEM observations but fall into the optimal size scope for high cellular uptake (Wina, et al., Biomaterials 2005, 26, 2713-272). The increased hydrodynamic size may be due to the formation of QD-FVIOs dimer or trimer in solution. In addition, similar to the PEI stabilized magnetite nanoparticles, the dispersion of QD-FVIOs is stable over a wide range of salt concentration and pH values due to the effective barrier and large buffering capacity of PEI (Goon, et al., Chem. Mater. 2009, 21, 673-681). FIGS. 4C and D show the influence of the salt concentration and pH value on relative PL intensity. We found that the QD-FVIOs maintained over 50% of their original fluorescence intensity when stored in 500 mM NaCl solution after 5 days or in hydrochloric acid solution with a pH of 3. The stable dispersion of QD-FVIOs, especially in acid environments, is of particular interest for the applications as intracellular imaging probes since most intracellular organelles such as endosomes and lysosomes are acidic with pH of 4-6.

### Magnetic and Optical Properties

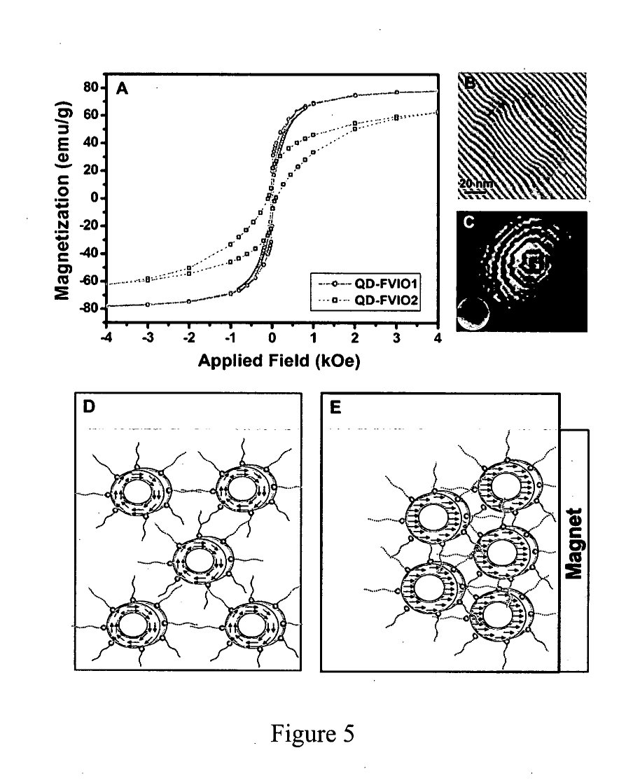

Magnetic characterizations of QD-FVIOs were carried out by using both SQUID magnetometer and TEM electron holography. As shown in FIG. 5A, the QD-FVIOs show high values of saturation magnetization of 72-78 emu/g, which is about 85% of that of bulk Fe₃O₄ (92 emu/g). Hysteresis loops of QD-FVIOs at 300 K show two distinct switching fields: the lower one at about 0.1 kOe and the other at a much higher field in the range of 1-3 kOe respectively, which corresponds to the transition from the onion state to the vortex state (Rothman, et al., Phys. Rev. Lett. 2001, 86, 1098-1101; and Zhu, et al., Phys. Rev. Lett. 2006, 96 027205). The presence of stable vortex state in QD-FVIOs has also been confirmed by electron holography. FIGS. 5B and C show an off-axis electron hologram and the corresponding magnetic induction map of a single QD-FVIO with an outer diameter of 92 nm and a height of 60 nm. The QD-FVIO shows a vortex state with minimal external stray fields where magnetic flux circulates around it. Therefore, the overall magnetic moment of each nanoring is zero in the absence of an external field and the magnetic interaction between the particles can be negligible for the water suspension of QD-FVIOs. SPIOs show a similar behavior (no magnetic interactions) due to the randomized magnetization because of the small size below the superparamagnetic limitation (10 nm). Under a small external field, the magnetization of QD-FVIOs will be quickly aligned along the field direction through a transition from the vortex state to the onion state and reach its maximum. The behaviors of vortex state and onion state in QD-FVIOs dispersion are schematically illustrated in FIGS. 5D and E, respectively. This property can also facilitate the delivery of these imaging nanoprobes to the targeted area of the body, such as cancerous tissues and tumors, via external magnetic manipulation.

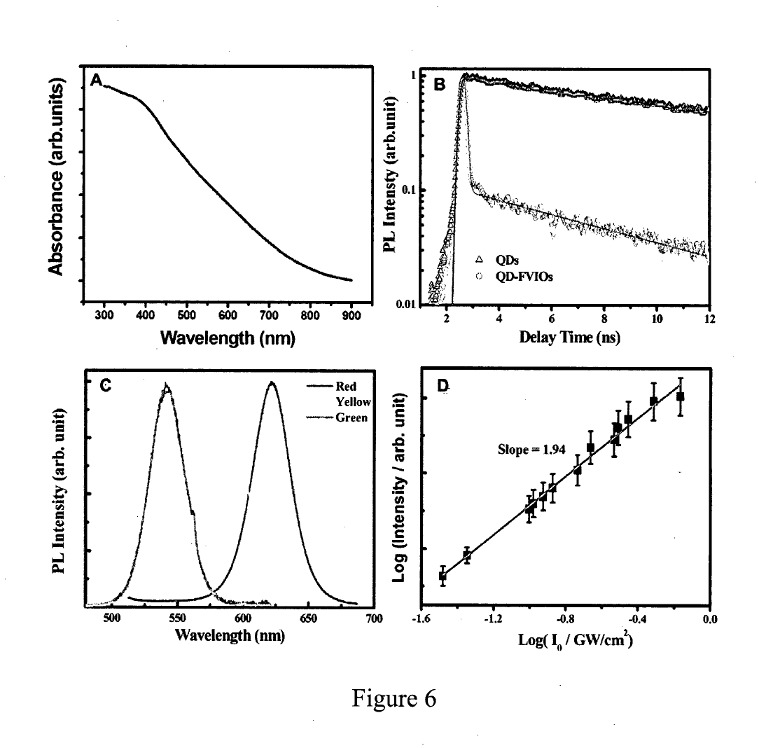

Optical properties of QD-FVIOs have been investigated by steady absorption and transient PL spectroscopy using a Coherent Legend regenerative amplifier. FIG. 6A shows a typical steady-state absorption spectrum of QD-FVIOs. There is no observable absorption band originating from QDs due to both the strong absorption of magnetite in UV-vis region and the low content of QDs. The typical transient PL data at the emission peak (±5 nm) for QD-FVIOs are given in FIG. 6B. For comparison, the PL dynamics in PEI-QDs dispersed in water were also measured under the same experimental conditions. The PL decay curve of isolated PEI-QDs was well described by a single exponential function with a time constant of 13 ns (Crooker, et al., Phys. Rev. Lett. 2002, 89, 186802). After conjugation, the QDs were closely packed on the surface of FVIOs and the quantum yields were also greatly reduced. The PL decay curves of QD-FVIOs can be fitted with two exponential functions. The fast decay component of about 0.02 ns dominates near 98% over the decay curve. This fast PL quenching is caused by the resonant energy transfer to FVIOs for the QDs in close contact with FVIOs. The conclusion is also supported by the other experimental reports that the lifetime of CDSe/ZnS QDs on Au substrate is less than 1 ns (Shimizu, et al., Phys. Rev. Lett. 2002, 89, 117401; and Ito, et al., Phys. Rev. B 2007, 75 033309). The slow decay component of about 7 ns originates from radiative recombination within closely packed QDs which are relatively far away from FVIQs. The slightly shortened lifetime compared with isolated PEI-QDs may be due to the dipole-dipole interaction between the neighboring QDs (Crooker, et al., Phys. Rev. Lett. 2002, 89, 186802). The upconversion PL spectra of multicolor QD-FVIOs excited by 800 nm laser pulse are shown in FIG. 6C, which are basically identical to the one-photon excitation PL spectra with narrow emission band (about 40 nm). FIG. 6D shows the nearly quadratic power dependence with a slope of 1.94 for corresponding PL signals. This quadratic power dependence under relatively low light excitation (<1 GW/cm2) confirms the two-photon absorption (2 PA) nature of QD-FVIOs (Xing, et al., Appl. Phys. Lett. 2008, 93, 241114).

### In Vitro MRI of QD-FVIOs

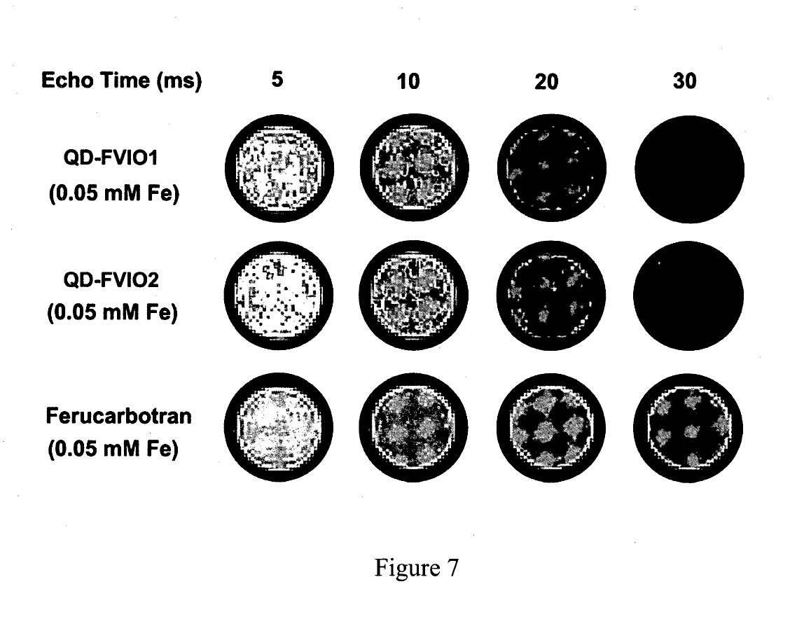

In vitro MRI of the OD-FVIOs was performed using a medical Siemens Symphony 1.5 T (61.8 MHz) scanner. FIG. 7 shows a qualitative comparison of T₂*-weighted spin-echo MRI of QD-FVIOs and commercial ferucarbotran with respect to the varied echo time (TE). Intensity values of QD-FVIO MR images shown in FIG. 7 have been adjusted for the T₂* effects of agarose relative to water. QD-FVIOs result in significantly greater signal reduction (darker images) at the designated TE from 10 to 30 ms in contrast to ferucarbotran. The MR relaxivities of QD-FVIOs and ferucarbotran are presented in Table 1.

TABLE 1
MR Relaxivities of QD-FVIOs and
Commercial Ferucarbotran at 1.5 T

| | r₁ | r₂ | | r₂* | |
|---|---|---|---|---|---|
| Sample | (s−1mM−1) | (s−1mM−1) | r₂/r₁ | (s−1mM−1) | r₂*/r₁ |
| QD-FVIO1 | 0.44 | 73.8 | 168 | 1079 | 2450 |
| QD-FVIO2 | 0.59 | 55.1 | 93 | 976 | 1654 |
| ferucarbotran | 11.3 | 225 | 19.9 | 254 | 22.5 |

The r₂* values of QD-FVIOs are almost quadruple that of the commercial ferucarbotran, while the r₂*/r₁ ratios are 2 orders of magnitude greater. Previous theoretical and experimental studies (Yablonskiy, et al., Magn. Reson. Med. 1994, 32, 749-763; and Na, et al., Adv. Mater. 2009, 21, 2133-2148) revealed that the r₂* relaxation rate strongly depends on the local field inhomogeneity which correlated with the relative volume fraction, magnetic moment of magnetic core, and susceptibility difference between particle and water. Hence, the extremely large r₂* relaxivity in QD-FVIOs obviously arises from its ring-like shape and magnetization process from vortex to onion that provide both high relative volume fraction and susceptibility. In addition, the internal field inhomogeneity of QD-FVIOs that originated from the susceptibility difference between the inner and outer surface of the magnetite nanoring may also contribute to the enhancement of the r₂* value. The r₁ and r₂ relaxivities of QD-FVIOs are less than that found for ferucarbotran (Table 1). As the enhancement of T₁ requires immediate contact between magnetic core and water molecules to effectively expedite spin-lattice relaxation (Qian, et al., Adv. Mater. 2007, 19, 1874-1878), the small r₁ relaxivity is understandable due to the surface chemistry of QD-FVIOs where magnetic core is segregated effectively from the exterior water molecules by a compact PEI layer. In addition, the r₂* value of QD-FVIO1 is only slightly higher (˜10%) than that of QD-FVIO2 despite its two times greater diameter. Moreover, both QD-FVIO1 and QD-FVIO2 show a much lower r₂ value as compared to ferucarbotran. These MR relaxation results (r₂* [symbol] r₂) are not consistent with that of the clustered SPIOs (Ai, et al., Adv. Mater. 2005, 17, 1949-1952) whose particle sizes typically display relaxation behavior in the motional averaging regime (MAR; r₂*=r₂) or near the transition of the MAR to the static dephasing regime (SDR; r₂−0.5−1×r₂*) wherein r₂* has reached a maximum (r₂*SDR) and does not increase further with particle diameter. The relaxation behaviors of QD-FVIOs (155 nm and 310 nm hydrodynamic diameter) might be expected to be near the MAR/SDR transition but appears rather to be more in agreement with that of larger particles whose relaxation behavior is said to be in a strongly echo-limited regime (ELR; r₂−0.05×r₂*) (Lee, et al., Biomaterials 2010, 31, 3296-3306). Increasing the particle size of FVIOs further would not give rise to significant enhancement of MR signals in spin-echo (i.e., T₂-weighted) sequences. FVIOs are of a more complex construction than SPIO aggregates, and it may be expected that the theory describing their MR relaxation behavior may be qualitatively different. It appears that further investigation of the effects of magnetic vortex core on T₂* relaxation time is warranted. Nevertheless, our results provide a new approach to achieve significant enhancement of the MRI signal in T₂* weighed sequences and using FVIO particles for potential cellular imaging applications.

### Multiphoton Fluorescence Imaging and Cell Uptake

The application of QD-FVIOs for two-photon fluorescence imaging in vitro was demonstrated using MGH bladder cancer cells. Upon incubation with the QD-FVIOs (50 μg/mL) in serum-free RPMI medium for 2 h at 37° C., the localized QD-FVIOs in the stained MGH cells can be brightly illuminated when imaged on the fluorescence microscope with excitation by 756 nm laser pulses. The yellow- and red-colored QD-FVIOs were able to label the cell membrane and the cytoplasm of MGH cells. The localization of QD-FVIOs in the cytoplasm indicates that these nanoparticles like PEI capped QDs (Duan, et al., J. Am. Chem. Soc. 2007, 129, 3333-3338) have escaped from the endosomes through the “proton sponge effect” and are released into the cytoplasm. More evidence of endosomal disruption and QD-FVIOs release comes from intracellular colocalization studies, in which QD-FVIOs and early endosome antigen 1 (EEA 1) are codelivered into living MGH cells. If the delivered QD-FVIOs are trapped in endosomes, their fluorescence signal will be colocalized with that of the EEA 1; on the other hand, if the QD-FVIOs are released into the cytoplasm, their fluorescence signal will not be colocalized with EEA 1. The colocalization can be readily detected when the QD-FVIOs and EEA 1 have different fluorescence colors. Significantly, diffused intracellular distribution for QD-FVIOs is observed. Moreover, TEM investigations of cellular uptake also confirm the escape of QD-FVIOs from endosomes. The QD-FVIOs are initially localized in vesicles after endocytosis, and then they disrupt the phospholipid membrane and escape from endosomes. Finally, the QD-FVIOs are slowly released into the cytoplasm. With the ability of cell penetration, these results suggest that these QD-FVIOs could be favored for intracellular imaging probes.

### Cytotoxicity Test

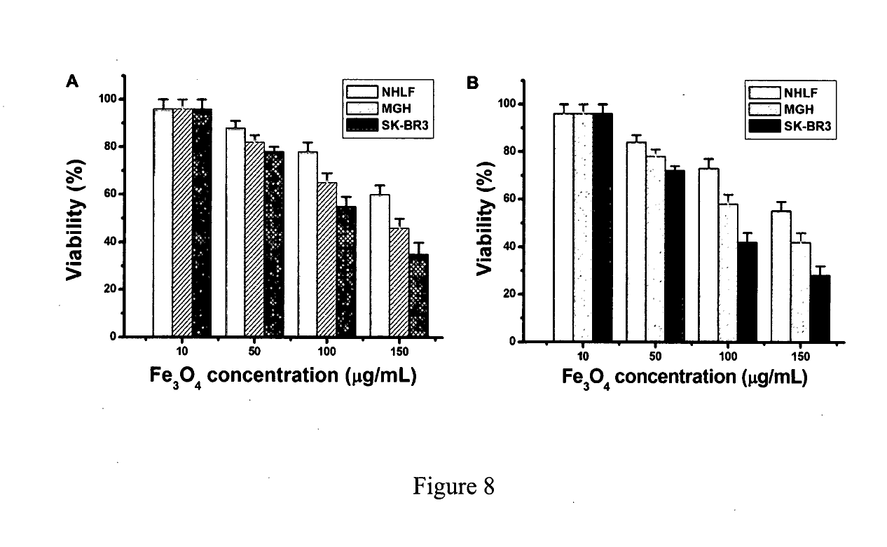

Although nanoparticle agents with cationic PEI coating are able to enhance cell uptake and penetration due to the electrostatic interactions with negatively charged glycocalyx on cell membranes, they are often associated with significant cytotoxic effects. It is thus important to evaluate the toxicity profiles of the QD-FVIOs using standard cytotoxicity tests. FIGS. 8A and B show the viability of the normal human lung fibroblast cells (NHLF), MGH bladder cancer cells, and SK-BR3 breast cancer cells after 24 h incubation with QD-FVIOs at 37° C. Both QD-FVIO1 and QD-FVIO2 show insignificant toxicity at low Fe₃O₄ concentration (<50 μg/mL) for all cells.

### In Vitro Hyperthermic Effects of Fe₃O₄ Nanorings

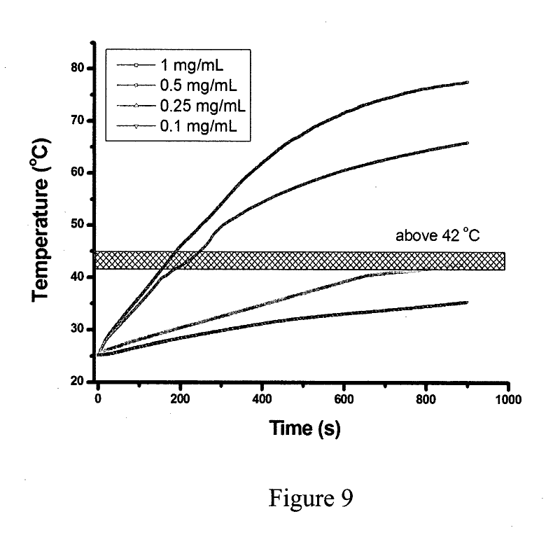

As shown in FIG. 9, the solution could be heated above 42° C. within 300 s if the concentration of Fe₃O₄ nanorings was above 0.25 mg/ml, e.g., 0.5 mg/ml and 1 mg/ml. These results suggest that Fe₃O₄ nanorings are effective as hyperthermic agents for cancer therapy.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

Further, any mechanism proposed in this disclosure does not in any way restrict the scope of the claimed invention.

What is claimed is:

1. A method of enhancing contrast of magnetic resonance imaging, the method comprising administering to a subject in need thereof an effective amount of an magnetic vortex core (MVC) nanoring.

2. The method of claim 1, wherein the MVC nanoring is 20-300 nm in height, 35-1000 nm in outer diameter, and 12-750 nm in inner diameter.

3. The method of claim 2, wherein the MVC nanoring is 20-150 nm in height, 35-250 nm in outer diameter, and 12-150 nm in inner diameter.

4. The method of claim 3, wherein the MVC nanoring is 70 or 160 nm in outer diameter.

5. The method of claim 1, wherein the MVC nanoring is a Fe₃O₄ or γ-Fe₂O₃ nanoring.

6. The method of claim 5, wherein the MVC nanoring is a Fe₃O₄ nanoring.

7. The method of claim 1, wherein the MVC nanoring is a quantum dot-capped MVC nanoring.

8. The method of claim 1, wherein the MVC nanoring is an MFe₂O₄ nanoring, M being a transition metal ion.

9. The method of claim 8, wherein M is a cobalt ion, copper ion, manganese ion, or nickel ion.

10. The method of claim 9, wherein M is a cobalt ion.

11. A method of treating tumor, comprising delivering an effective amount of a MVC nanoring to a tumor site in a subject, placing the tumor site in an alternating magnetic field (AMF), and maintaining the tumor site in the AMF for a pre-determined duration of time so as to kill tumor cells.

12. The method of claim 11, wherein the AMF has a frequency of 100-500 kHz.

13. The method of claim 12, wherein the MVC nanoring is 20-300 nm in height, 35-1000 nm in outer diameter, and 12-750 nm in inner diameter.

14. The method of claim 13, wherein the MVC nanoring is 20-150 nm in height, 35-250 nm in outer diameter, and 12-150 nm in inner diameter.

15. The method of claim 14, wherein the MVC nanoring is 70 or 160 nm in outer diameter.

16. The method of claim 11, wherein the MVC nanoring is a Fe₃O₄ or γ-Fe₂O₃ nanoring.

17. The method of claim 16, wherein the MVC nanoring is a Fe₃O₄ nanoring.

18. The method of claim 11, wherein the MVC nanoring is a quantum dot-capped MVC nanoring.

19. The method of claim 11, wherein the MVC nanoring is an MFe₂O₄ nanoring, M being a transition metal ion.

20. The method of claim 19, wherein M is a cobalt ion, manganese ion, or nickel ion.

21. The method of claim 20, wherein M is a cobalt ion.

22. The method of claim 11, wherein the method is combined with radiotherapy or chemotherapy.
